(19) United States
(12) Patent Application Publication
(10) Pub. No.: US 2026/0103677 A1
(43) Pub. Date: Apr. 16, 2026

(54) Inhibition of microbial methanogenesis

(71) Applicant: The Regents of the University of California, Oakland, CA (US)

(72) Inventor: Hans Karl Carlson, Berkeley, CA (US)

(73) Assignee: The Regents of the University of California, Oakland, CA (US)

(21) Appl. No.: 19/409,631

(22) Filed: Dec. 4, 2025

Related U.S. Application Data
(63) Continuation of application No. PCT/US24/35164, filed on Jun. 23, 2024.
(60) Provisional application No. 63/522,725, filed on Jun. 23, 2023.

(51) Int. Cl. C12N 1/205; C05B 17/00; C12N 9/02; C12N 15/82; C12R 1/01

(52) U.S. Cl. CPC C12N 1/205; C05B 17/00; C12N 9/0069; C12N 15/8243; C12R 2001/01; C12Y 113/11

(57) ABSTRACT

Hypophosphite is used to inhibit syntrophic methanogenesis or growth of hydrogenotrophic methanogens, plants are genetically engineered to comprise and express a heterologous hypophosphite/2-oxoglutarate dioxygenase htxa gene to enable the use of the reduced phosphorus, such as hypophosphite as a phosphorus source, and hypophosphite and phosphite fertilizer amendments are utilized as phosphorus sources by soil microorganisms, to increase carbon storage in deep sediments.

### CROSS-REFERENCES TO RELATED APPLICATIONS

This application is a continuation of PCT/US24/35164, filed: Jun. 23, 2024, which claims priority to U.S. Provisional Application No. 63/522,725, filed: Jun. 23, 2023, the disclosures of which are hereby incorporated by reference in its entirety for all purposes.

### GOVERNMENT SUPPORT CLAUSE

This invention was made with government support under Grant Number DE-AC02-05CH11231 awarded by the United States Department of Energy. The government has certain rights in the invention

### INTRODUCTION

Microbial methanogenesis is responsible for the majority of emissions of the greenhouse gas methane into the atmosphere (Rosentreter et al. 2021). However, currently known inhibitors of microbial methanogenesis are limited in number, non-specific and require high concentrations to be effective (Liu et al. 2011). Nevertheless, the methanogenesis pathway is highly conserved and specific to methanogens and thus should be highly druggable. Commonly used inhibitors of methanogenesis include compounds such as bromoethanesulfonic acid (BES), 3-nitroxypropanol (3-NOP) lumazine and halogenated analogs of methane or acetate (Liu et al. 2011). These compounds vary in their inhibitor potency, are costly to produce and are not viable for application in many environmental methanogenic systems.

Microbial methanogenesis in environmental systems involves syntrophic degradation of complex organic carbon by different bacterial sub-populations that sequentially depolymerize and then ferment complex organic carbon to produce organic acids that are subsequently converted by syntrophic bacteria into hydrogen, formate and acetate which serve as the primary electron donors for methanogenic archaea (FIG. 1). Syntrophic conversion of organic acids is thermodynamically favorable when hydrogen, formate and acetate concentrations are maintained at low concentrations by the activity of methanogens. Thus, syntrophic bacteria and methanogenic archaea are found in close association in methane producing natural ecosystems and inhibition of either population will inhibit methane production (FIG. 1).

Hypophosphite is a formate analog and competitive inhibitor of formate metabolic enzymes (Takamiya 1953). Furthermore, hypophosphite is an inhibitor of co-cultures of syntrophic butryate oxidizers and hydrogenotrophic methanogens when formate is the metabolic intermediate (Sieber, Le, and McInerney 2014). In complex syntrophic communities, formate, hydrogen and acetate are all important syntrophic products used by distinct classes of methanogens. Inhibition of formate metabolism will feed back on the syntrophic community to inhibit it which will in turn lead to accumulation of organic acids to ultimately shut down the entire methanogenic system. In other studies, inhibition of acetate utilizing methanogenesis by monofluoroacetate was shown to inhibit methanogenesis despite the likelihood that hydrogenotrophic and methylotrophic methanogens were also present in the complex methanogenic test environments (Emptage, Tabinowski, and Odom 1997).

Previous work did not consider nor assess the potency or specificity of hypophosphite as an inhibitor of methanogenesis. Furthermore, previous work studying the impact of hypophosphite on methanogens examined simple co-cultures of methanogenic archaea and syntrophs and did not consider complex microbial communities, e.g. Sieber, Jessica R., Huynh M. Le, and Michael J. McInerney. 2014. “The Importance of Hydrogen and Formate Transfer for Syntrophic Fatty, Aromatic and Alicyclic Metabolism.” Environmental Microbiology 16 (1): 177-88.

Relevant Literature includes: Pine 1970 The Methane Fermentations, in: Anaerobic Biological Treatment Processes, Chapter 1, p1-10, Advances in Chemistry, Vol. 105.

### SUMMARY OF THE INVENTION

In an aspect, the invention provides a method of inhibiting microbial methanogenesis, comprising use of a formate analog, hypophosphite, to inhibit syntrophic methanogenesis or growth of hydrogenotrophic methanogens of a complex, environmental microbial community.

In embodiments:
the complex, environmental microbial community is present in a crop field, wastewater, bioprocessing system, such as bioreactors for renewable natural gas (RNG, or natural or managed ecosystem; and/or
the method further comprising using the inhibition of methanogenesis to obtain carbon credits.

In an aspect, the invention provides a method of engineering of hypophosphite assimilating crop (e.g. rice) plants to increase the efficiency of phosphorus fertilizer amendments in crop fields while mitigating methane emissions, comprising genetically engineering a plant to comprise and express a heterologous hypophosphite/2-oxoglutarate dioxygenase htxa gene to enable the use of the reduced phosphorus, such as hypophosphite as a phosphorus source.

In embodiments, the crop plant is rice, corn, wheat, yam, soybean, sugarcane or cotton.

In an aspect, the invention provides a method using reduced phosphorus compounds as P fertilizer for deep soil carbon sequestration, comprising use of a reduced phosphorus compound like hypophosphite and phosphite fertilizer amendments which are more mobile in soils and sediments, but can be utilized as phosphorus sources by soil microorganisms, to increase carbon storage in deep sediments.

In embodiments the compound is hypophosphite, the soil is of a rice crop field, wherein the delivered hypophosphite is stable for >6 months in anaerobic sediment.

The invention encompasses all combinations of the particular embodiments recited herein, as if each combination had been laboriously recited.

### BRIEF DESCRIPTION OF THE DRAWINGS

FIG. 1. Carbon and electron flow in anaerobic methanogenic systems. Arrows indicate fluxes inhibited by an inhibitor of formate metabolism as presented here.

FIG. 2. Dose response of hypophosphite inhibition of Methanococcus maripaludis S2 grown with either 150 mM formate (grey) or 80% hydrogen (black) as the sole electron donor.

FIG. 3. Dose response of hypophosphite inhibition of growth and metabolism of nitrate, sulfate and methanogenic enrichment cultures from rice field sediment with yeast extract as the sole electron donor and carbon source. Open symbols represents the bulk growth as measured by optical density (OD) of the fermentative populations in each enrichment and closed symbols represent production of nitrate, sulfide or methane as proxies for each metabolic activity. The IC50 with 95% confidence interval against methane production in the methanogenic cultures is 56 (39-78) micromolar.

FIG. 4. Dose response of hypophosphite inhibition of growth, methanogen abundance and methane production relative to un-treated controls in methanogenic enrichment cultures from rice field sediment with yeast extract as the sole electron donor and carbon source. Open triangles represents the bulk growth as measured by optical density (OD) of the fermentative populations in each enrichment, closed triangles represent production methane and closed squares represent releative abundance of methanogenic archaea as measured by 16S rDNA amplicon sequencing The IC50s with 95% confidence intervals against methane production is 56 (39-78) micromolar and against methanogen abundance is 136 (109-179) micromolar.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### Example 1. Inhibition of Microbial Methanogenesis by Hypophosphite

Hypophosphite (P in the +1 oxidation state, H₂PO²⁻), and phosphite (P in the +3 oxidation state, HPO³⁻) is not available as a phosphorus source for plants or animals which can only use phosphorus as phosphate (P in the +5 oxidation state, PO₄²⁻). Both phosphite and hypophosphite must be oxidized to phosphate to serve as phosphorus sources and to be incorporated into biomass. The only known pathways for reduced phosphorus oxidation are microbial. Anaerobic phosphite oxidation pathways exist, but for hypophosphite only oxygen-requiring pathways are known. Thus, phosphite can be a phosphorus source for both aerobic and anaerobic bacteria, but hypophosphite can only serve as a phosphorus source for aerobic bacteria. Hypophosphite is a formate analog and an inhibitor of methanogenesis. It will be most persistent in anaerobic soils.

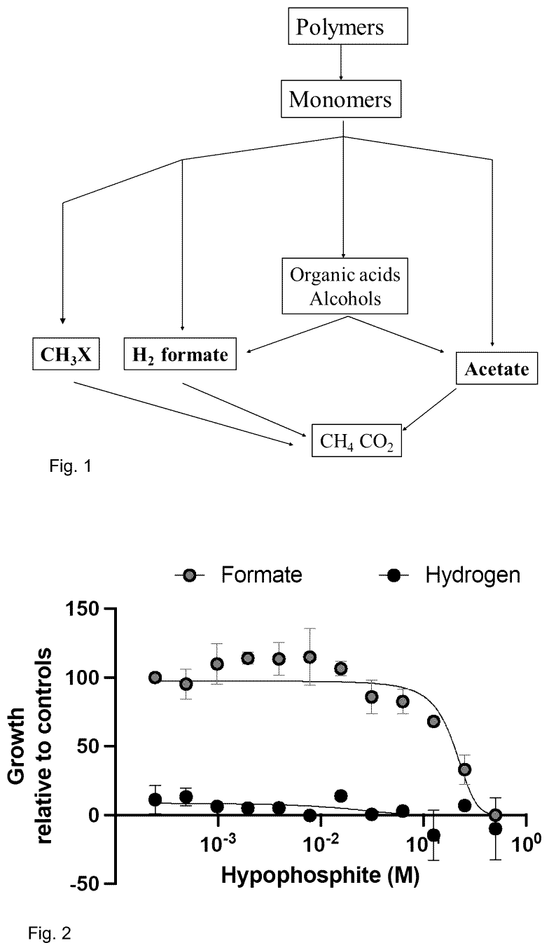

Our invention preferably employs the formate analog, hypophosphite, to inhibit syntrophic methanogenesis (FIG. 1). We have performed laboratory tests to demonstrate selectivity and potency of hypophosphite in diverse methanogens and methanogenic systems. We disclose and our results demonstrate that hypophosphite is extremely inhibitory of hydrogenotrophic methanogens that produce trace formate for biosynthetic reactions (FIG. 2). While hypophosphite inhibits growth of the S2 culture with 160 mM formate at concentrations >100 mM, hypophosphite is inhibitory at concentrations below 0.1 mM when S2 is cultured on hydrogen.

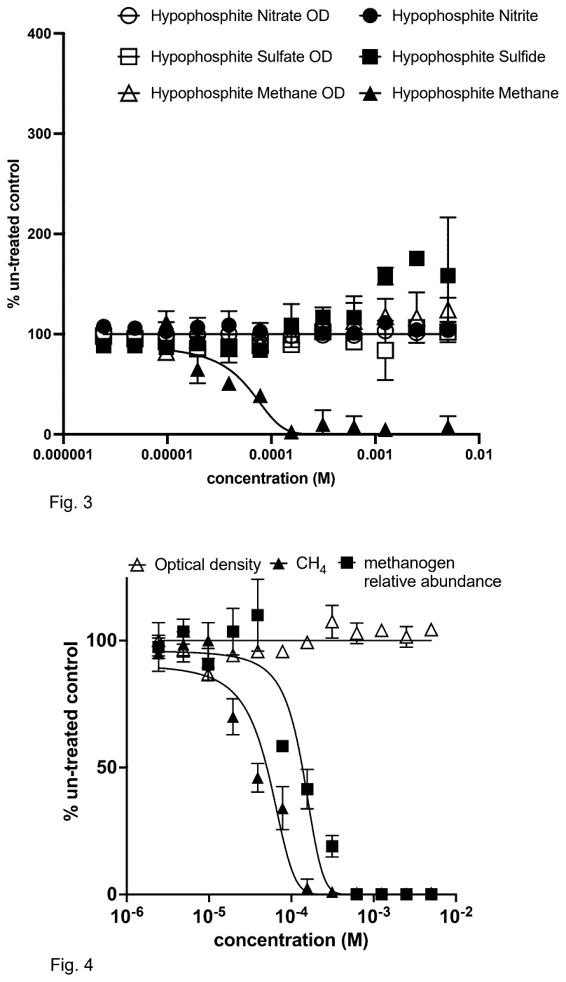

In a complex methanogenic community from rice field soil, we also disclose and demonstrate that hypophosphite is selectively inhibitory of methanogenesis versus fermentative growth, nitrate reduction or sulfate reduction (FIG. 3). We have also demonstrated that not only is methanogenesis inhibited in the context of the rice field microbiome by hypophosphite but also the growth of methanogenic archaea (FIG. 4). This selectivity is important for application as the therapeutic window for hypophosphite application to enable shifting of metabolism towards other processes other than methanogenesis is wide.

Practical applications include: agricultural industry, wastewater industry, bioprocess industry, managed ecosystems, and carbon credits. Significantly, hypophosphite is already a safe and effective fertilizer additive and is approved for use in diverse agricultural systems and is found in diverse environmental systems, as it is produced naturally by environmental bacteria (Hanrahan et al. 2005).

### References

Emptage, Mark, J. Tabinowski, and J. Martin Odom. 1997. “Effect of Fluoroacetates on Methanogenesis in Samples from Selected Methanogenic Environments.” nvironmental Science & Technology31 (3): 732-34.
Hanrahan, Grady, Tina M. Salmassi, Crist S. Khachikian, and Krishna L. Foster. 2005. “Reduced Inorganic Phosphorus in the Natural Environment: Significance, Speciation and Determination.” Talanta66 (2): 435-44.
Liu, He, Jin Wang, Aijie Wang, and Jian Chen. 2011. “Chemical Inhibitors of Methanogenesis and Putative Applications.” pplied Microbiology and Biotechnology89 (5): 1333-40.
Rosentreter, et al. 2021. “Half of Global Methane Emissions Come from Highly Variable Aquatic Ecosystem Sources.” Nature Geoscience14 (4): 225-30.
Sieber, Jessica R., Huynh M. Le, and Michael J. McInerney. 2014. “The Importance of Hydrogen and Formate Transfer for Syntrophic Fatty, Aromatic and Alicyclic Metabolism.” Environmental Microbiology16 (1): 177-88.
Takamiya, Atusi. 1953. “Studies on the Formic Dehydrogenase of Escherichia Coli III. Determination of the Quantity of the Enzyme within the Cell by Using Hypophosphite as a Specific Inhibitor.” Journal of Biochemistry40 (4): 407-14.

### Example 2. Engineering of Hyposphosphite Assimilating Rice Plants to Increase the Efficiency of Phosphorus Fertilizer Amendments in Rice Fields while Mitigating Methane Emissions

This aspect of the invention provides engineering of plants to possess the hypophosphite oxidase (htxA) gene to enable the use of the reduced phosphorus compound as a phosphorus source. This invention is particularly applicable to rice agriculture; see, e.g. the use of hypophosphite as an inhibitor or microbial methogenesis, herein.

Phosphite has been used as a fertilizer and pesticide combination in agricultural crops that are engineered to utilize phosphite as a phosphorus source including rice plants (Lovatt and Mikkelsen 2006; Havlin and Schlegel 2021; Manna et al. 2016). This is accomplished by introduction of the bacterial ptxD phosphite dehydrogenase gene. Phosphite tends to be more mobile in soils (Zhu, Carlson, and Coates 2013) and have a longer half-life compared to phosphate. This is similarly the case for hypophosphite, which we have also shown to be effective for months in inhibiting methanogenesis in batch methanogenic incubations in our laboratory and to be essentially non-utilizable as a phosphorus source in anaerobic gut environments (Rhodehamel, Pierson, and Leifer 1990). Thus, hypophosphite can provide a selective phosphorus source to rice plants that would not be utilized by the plant microbiome while simultaneously inhibiting methanogenesis.

The present invention describes the approach of introducing the hypophosphite/2-oxoglutarate dioxygenase htxa gene (White and Metcalf 2002) into rice plants using well established transformation protocols (Toki et al. 2006). Resultant transgenic rice plants, similar to their phosphite utilizing analogs, are capable of utilization of hypophosphite as a phosphorus source.

We have shown hypophosphite to be a selective inhibitor of methanogenesis, rice plants are readily transformable and the ptxD phosphite dehydrogenase has already been shown work in rice plants. Here we disclose that introduction of a htxA hypophosphite dehydrogenase gene confers the ability of rice to utilize hypophosphite. In the event that hypophosphite is used for methane inhibition this also allows the P in in hypophosphite to be used to support plant growth; i.e. provide dual benefits to rice plants by providing hypophosphite as a dual methane inhibitor/phosphorus source.

Hypophosphite is as cost-effective as phosphite, and more stable in anaerobic ecosystems and able to inhibit methanogenesis in rice plants. Practical applications include agricultural clients/carbon sequestration.

### References

Havlin, John L., and Alan J. Schlegel. 2021. “Review of Phosphite as a Plant Nutrient and Fungicide.” Soil Systems 5 (3): 52.
Lovatt, C. J., and R. L. Mikkelsen. 2006. “Phosphite Fertilizers: What Are They? Can You Use Them? What Can They Do? ” 2006. https://spectrumanalytic.com/support/library/pdf/Phosphite_Fertilizers_What are they.pdf.
Manna, et al. 2016. “The Development of a Phosphite-Mediated Fertilization and Weed Control System for Rice.” Scientific Reports 6 (April): 24941.
Rhodehamel, E. J., M. D. Pierson, and A. M. Leifer. 1990. “Hypophosphite: A Review.” Journal of Food Protection 53 (6): 513-18.
Toki, et al. 2006. “Early Infection of Scutellum Tissue with Agrobacterium Allows High-Speed Transformation of Rice.” The Plant Journal: For Cell and Molecular Biology 47 (6): 969-76.
White, et al. “Isolation and Biochemical Characterization of hypophosphite/2-Oxoglutarate Dioxygenase. A Novel Phosphorus-Oxidizing Enzyme from Psuedomonas Stutzeri WM88.” The Journal of Biological Chemistry 277 (41): 38262-71.

### Example 3. Use of Reduced Phosphorus Compounds as P Fertilizer for Deep Soil Carbon Sequestration

This aspect of the invention provides a method of increasing carbon storage in deep sediments through the use of hypophosphite and phosphite fertilizer amendments which are more mobile in soils and sediments, but can be utilized as phosphorus sources by soil microorganisms.

Increasing carbon storage capacity of agricultural lands could be a nature-based solution to combat climate change. Generation of stable carbon by stimulating microbial growth in deep soils has been proposed as an approach to achieve this goal. The half-life of deep carbon is generally much longer than shallow carbon. Unfortunately, in many deep soils and sediments (Jørgensen and Marshall 2016), phosphorus and nitrogen are limiting and, as such, much of the microbial carbon is respired to carbon dioxide and methane. This limits the capacity of microbial biomass to accumulate and adds to the greenhouse gas emissions from soils and sediments. Microbial respiratory metabolism likely proceeds to provide energy to maintain biomass (Finstad et al. 2023), Designing fertilizer amendments to provide phosphorus and nitrogen to deep soils could circumvent this problem.

Orthophosphate is the most common fertilizer used in agricultural systems. It is bioavailable, but at neutral pH is a dianion and forms tight complexes with divalent cations such as Ca, Mg and Fe which are common in soils. As such, it tends to be depleted in deeper sediments. In contrast, phosphite and hypophosphite are monoanionic and much more soluble in the presence of divalent cations. We have shown that even in high iron sediment columns, phosphite is able to remain in solution to serve as a phosphorus source for microbial growth (Zhu, Carlson, and Coates 2013). Hypophosphite, like phosphite, is very soluble and has the additional benefit that it is a selective inhibitor of methanogenesis which has 10-fold the greenhouse forcing of carbon dioxide and is likely to be produced in high carbon subsurface environments when other terminal electron acceptors are depleted.

Both hypophosphite and phosphite are extremely water soluble, and may be applied as any other phosphorus fertilizer. Unlike phosphate they will not bind as tightly to soil cations, so they will migrate deeper into soils, e.g. >6, 12, 18, 24 or 36″. In embodiments, hypophosphite is delivered to rice fields, ruminants or reactors to inhibit methanogenesis. All of these are anaerobic systems where the hypophosphite will persist for long periods of time, e.g. >6 months.

There is no prior plant method to use hypophosphite (only phosphite). The targeting of deep soils and sediments with hypophosphite is novel. Prior work has used phosphite surface delivery for weed suppression. The invention is also distinct from plant nutrition or fertilizing, and while the added benefit of methanogenesis inhibition applies, the target result is maintaining a more soluble form of phosphorus to support deep soil carbon sequestration. The concentrations and formulations are adapted accordingly. Indeed, delivery to plants may also support microbial biomass, however, there is no known mechanism of anaerobic hypophosphite oxidation so in flooded rice field there would not be enough oxygen available to support hypophosphite oxidation.

Our ongoing work on the field validation in rice and ruminants and reactor systems support efficacy of hypophosphite. For example, we have demonstrated that hypophosphite is stable for >6 months in anaerobic sediments; see, e.g. FIG. 2.

### References

Finstad, et al. 2023. “Radiocarbon analysis of soil microbial biomass via direct chloroform extraction.” Radiocarbon, Oct, 1-9.
Jørgensen, Bo Barker, and Ian P. G. Marshall. 2016. “Slow Microbial Life in the Seabed.” Annual Review of Marine Science 8:311-32.
Zhu, Hongbo, Han K. Carlson, and John D. Coates. 2013. “Applicability of Anaerobic Nitrate-Dependent Fe(II) Oxidation to Microbial Enhanced Oil Recovery (MEOR).” Environmental Science & Technology 47 (15): 8970-77.

1. A method of inhibiting microbial methanogenesis, comprising use of a formate analog, hypophosphite, to inhibit syntrophic methanogenesis or growth of hydrogenotrophic methanogens of a complex, environmental microbial community.

2. The method of claim 1, wherein the complex, environmental microbial community is present in a crop field, wastewater, bioprocessing system, or natural or managed ecosystem.

3. The method of claim 1, further comprising using the inhibition of to obtain carbon credits.

4. The method of claim 2, further comprising using the inhibition of to obtain carbon credits.

5. A method of engineering of hypophosphite assimilating crop plants to increase the efficiency of phosphorus fertilizer amendments in crop fields while mitigating methane emissions, comprising genetically engineering a plant to comprise and express a heterologous hypophosphite/2-oxoglutarate dioxygenase htxa gene to enable the use of the reduced phosphorus, such as hypophosphite as a phosphorus source.

6. The method of claim 5, wherein the crop plant is rice, corn, wheat, yam, soyabean, sugarcane or cotton.

7. A method using reduced phosphorus compounds as phosphorous (P) fertilizer for deep soil carbon sequestration, comprising use of a reduced phosphorus compound like hypophosphite and phosphite fertilizer amendments which are more mobile in soils and sediments, but can be utilized as phosphorus sources by soil microorganisms, to increase carbon storage in deep sediments.

8. The method of claim 7, wherein the compound is hypophosphite, the soil is of a rice crop field, wherein the delivered hypophosphite is stable for >6 months in anaerobic sediment.
